(19) United States
(12) Patent Application Publication
(10) Pub. No.: US 2009/0285882 A1
(43) Pub. Date: Nov. 19, 2009

(54) Stabilized Liposome Compositions and Related Methods of Use

(76) Inventors: Jochen Weiss, Stuttgart (DE); David Julian McClements, Northampton, MA (US); Eric Decker, Sunderland, MA (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 12/428,268

(22) Filed: Apr. 22, 2009

Related U.S. Application Data
(60) Provisional application No. 61/125,014, filed on Apr. 22, 2008.

(51) Int. Cl. A61K 9/127

(52) U.S. Cl. 424/450

(57) ABSTRACT

Liposome compositions and related methods of preparation which can comprise one or a plurality of biopolymer components.

This application claims priority benefit from application Ser. No. 61/125,014 filed Apr. 22, 2008, the entirety of which is incorporated herein by reference.

### BACKGROUND OF THE INVENTION

Liposomes or lipid vesicles are typically formed from polar lipids that are available in abundance in nature. Sources of raw material include soy and egg-derived phospholipids. Liposomes have shown to be able to incorporate a wide variety of functional components in their interior. They have proven to be of interest to a wide variety of industries including the pharmaceutical, chemical and food industries as carriers for both water- and lipid-soluble compounds. Liposomes are spherical, polymolecular aggregates with a bilayer shell configuration. Depending on the method of preparation, lipid vesicles can be multi- or unilamellar, containing many, or a single bilayer shell(s), respectively (FIG. 1).

Liposomes typically vary in size between 20 nm and a few hundred micrometers. Liposome cores are aqueous in nature, the chemical composition of which correspond to that of the range of aqueous solution in which such vesicles can be prepared. Because of the charge of the polar lipids used in their preparation, water soluble polar or ionic species can be entrapped inside the liposome cores. Based on the ability to effectively block penetration of charged species, liposomes have been successfully used to, for example, encapsulate proteins and provide a microenvironment in which proteins can continue to function regardless of external environmental conditions. On the other hand, the interior of the bilayer (i.e., the region where the hydrophobic tails of, for instance, the phospholipids directly interact) has properties that resemble that of an organic solvent and consequently, lipid compounds can be encapsulated between and within the bilayer, a process known as adsolubilization. Moreover, the lipid bilayer can serve as a support phase to for example include antibodies to provide an effective targeting system.

Many food applications employ liposomes. For example, liposomes can increase shelf life of dairy products by encapsulating lactoferrin, a bacteriostatic glycoprotein as well as nisin Z, an antimicrobial polypeptide. Liposomal entrapped phosvitin has been used to inhibit lipid oxidation in a variety of dairy products and ground pork. Liposome encapsulated vitamin C retained 50% activity after 50 days of refrigerated storage compared to free ascorbic acid which lost all activity after 19 days. Likewise, related studies demonstrated the ability of liposomes to act as carrier of antimicrobials to improve food safety. In these studies, long-term activity of antimicrobials, such as nisin encapsulated in the bilayer structure, was greatly prolonged and food products remained unspoiled for an extended period of time—thus increasing the shelf life of these products.

However, one of the major current limitations of liposomes is a tendency to “leak”; that is, lose core- or bilayer-encapsulated content over time. For example, if the encapsulated compound is an antimicrobial, the ability to inhibit growth of pathogens or spoilage organisms can decrease over time. If the encapsulated compound is a chemically susceptible bioactive compound, the bioactivity and bioavailability may decrease if it leaks into the exterior phase. To date, improved stability has focused on optimizing manufacturing procedures, modifying lipidic component (e.g., a phosphilipid) composition and adding stabilizing sugars to the inner or outer phase. Another limitation is that interaction of the liposomes with other components in the solvent phase is predominantly governed by the bilayer composition, i.e., the type and concentration of the lipidic components that comprise the membrane of the liposome. Finally, liposomes, depending on the strength of their bilayer construction, are susceptible to disruption by mechanical forces, during preparation and subsequent application/use, that lead to formation of pores and loss of encapsulated content.

To address such interactions, a modification of the liposome composition is typically required. For example, phospholipids may be used that are either positively or negatively charged to alter the charge of the hydrophilic peripheral surface component of the liposome. Such a tailored liposome would have a profoundly different interaction with other charged components in the aqueous phase. Specifically, inclusion of positively charged phospholipids in liposomes carrying antimicrobials may result in a liposome that is more antimicrobially active due to improved interaction with the membrane of microorganisms. However, use of purified phospholipids required for such a modification is expensive and, as a result, is neither industrially nor commercially practical.

As discussed above, there remains an on-going search in the art for improved liposome composition and function. An alternate approach is needed to better utilize the benefits and advantages available through liposome encapsulation and delivery of various active agents.

### SUMMARY OF THE INVENTION

In light of the foregoing, it is an object of the present invention to provide liposome compositions and/or methods for their stabilization, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It can be an object of the present invention to provide a liposome composition stabilized with respect to loss or degradation of encapsulated content.

It can be another object of the present invention to provide stabilized liposome compositions using readily available, regulatory-approved ingredient components.

It can be another object of the present invention, alone or in conjunction with one or more of the preceding objectives, to provide a facile, economic route for the preparation of such stabilized liposome compositions, as compared to various approaches of the prior art.

In part, the present invention can be directed to a stabilized liposome composition. Such a composition can comprise a liposome comprising a lipidic compound, such a liposome comprising a hydrophilic peripheral surface component, at least a portion of which can have a net charge; and a first bipolymer component thereabout, at least a portion of which can have a net charge opposite the net charge of the liposome peripheral surface component. In certain embodiments, such a lipidic compound can be selected from phospholipid compounds, phosphatide compounds and combinations of such compounds. Regardless, without limitation, such a first bipolymer component can be selected from proteins, carbohydrates and combinations thereof.

Without limitation, the surface component of such a liposome can comprise a net negative charge, and such a first bipolymer component can comprise a net positive charge. In certain non-limiting embodiments, such a composition can comprise a soy lecithin liposome and a chitosan layer thereabout. As illustrated therewith, stabilized liposome compositions of this invention can comprise lipidic compounds and first bipolymer components.

Various embodiments of such a composition can comprise a second bipolymer component about a first bipolymer component, at least a portion of the second bipolymer component comprising a net charge opposite the net charge of the first bipolymer component. In certain such embodiments, a first bipolymer component can have a net positive charge, and such a second bipolymer component can have a net negative charge. In certain such non-limiting embodiments, such a composition can comprise a chitosan or whey protein isolate first bipolymer component and a pectin second bipolymer component layered thereabout. Likewise, as discussed above and illustrated elsewhere herein, each such bipolymer component and liposome component can be food grade and/or generally recognized as safe for human use or consumption.

Without limitation, a first or second bipolymer component can comprise any polymeric material capable of adsorption on, electrostatic interaction with and/or coupling to a liposome and/or a charged surface component thereof. Accordingly, such a bipolymer component can be selected from one or more proteins, one or more polysaccharides, synthetic polymers and combinations thereof. Without limitation, such a bipolymer component can be selected from but not limited to ionic or ionizable proteins such as whey, casein, soy, egg, plant, meat and fish proteins, ovalbumins, glycoproteins, mucoproteins, phosphoproteins, serum albumins and collagens, and ionic or ionizable polysaccharides such as chitosan and/or chitosan sulfate, cellulose, pectins, alginic acids, alginates, nucleic acids, glycogen, amylose, chitin, polynucleotides, gum arabic, gum acacia, carageenans, xanthans, agars, tree gums and exudates thereof, guar gum, gellan gum, tragacanth gum, karaya gum, locust bean gum, lignin and/or combinations thereof. As mentioned above, such protein components can be selected on the basis of their amino acid residues (e.g., lysine, arginine, aspartic acid, glutamic acid, etc.) to optimize overall net charge, interaction with a layer component and/or resultant liposome stability. The polymeric component may alternatively be selected from modified polymers such as modified starch, modified celluloses, carboxymethyl cellulose, carboxymethyl dextran or lignin sulfonates.

Without limitation as to lipidic compound identity, a liposome composition of this invention can comprise a configuration selected from unilamellar and multilamellar bilayer configurations. Regardless of any such configuration, such a bilayer can comprise one or more active agents at least partially-soluble and/or miscible therein. Likewise, regardless of any such configuration, a liposome core can comprise an aqueous medium comprising one or more active agents (e.g., without limitation pharmaceutical, nutritional, antimicrobial, etc.), one or more of such agents at least partially soluble and/or miscible therein. Accordingly, depending upon active agent(s), such a stabilized liposome composition can be incorporated into a corresponding end-use product or material.

In part, the present invention can also be directed to a method of using a bipolymer component to stabilize a liposome composition. Such a method can comprise providing a liposome comprising a surface component comprising a net charge; and contacting such a liposome with a bipolymer component, where at least a portion of the bipolymer component has a net charge opposite the surface component net charge. The extent of such contact can vary with bipolymer and/or depending upon desired end result. Without limitation, as illustrated more fully below, where a liposome has a ζ-potential of one charge (e.g., negative or positive), a bipolymer component can be present in an amount at least partially sufficient to provide a resulting composition with a ζ-potential of an opposite charge (e.g., positive or negative, respectively) under conditions employed.

Without limitation and as applicable to various compositional aspects of this invention, a liposome can be selected from and/or comprise one or more lipidic compounds (e.g., phospholipids, phosphatides, etc.) known in the art. Regardless, a bipolymer component useful with such a methodology can be selected from proteins, polysaccharides and combinations thereof.

In certain embodiments of such a methodology, net-charged liposomes can be contacted with one or more bipolymer components comprising an opposite net charge, for direct adsorption, interaction and/or coupling therewith. Alternatively, in certain other embodiments, a bipolymer component can be incorporated or in contact with a liposome under conditions or at a pH not conducive for sufficient adsorption, electrostatic interaction and/or coupling therewith. The pH can then be varied and/or adjusted to change the net electrical charge of the liposome, a surface component thereof, and/or of a bipolymer component, sufficient to promote electrostatic interaction with and incorporation of the bipolymer component. In certain such embodiments, a bipolymer component can comprise a protein, and pH can be lowered below the isoelectric point of such a protein to promote desired interaction.

In certain other embodiments, regardless of net charge, origin or adjustment, such a first bipolymer component can be contacted with another or subsequent second bipolymer component, wherein at least a portion of the other, subsequent second bipolymer component has a net charge opposite the net charge of the first bipolymer component contacting the liposome. Iterative and/or sequential contacts, adsorption, electrostatic interactions and/or couplings of oppositely-charged bipolymer (e.g., such first and second biopolymer components, various other biopolymer components and combinations thereof) components can be used to incrementally coat a liposome with multiple (e.g., without limitation, 2, 3, 4 . . . about 5 . . . about 7 or more) biopolymer components or layers to afford functional (e.g., charge or permeability) and structural (e.g., component or multi-component thickness) benefits of the sort described elsewhere herein.

Whether or not such a methodology is engaged in an aqueous medium, the resulting liposome compositions can be isolated, as would be understood by those skilled in the art, for further use or application, including but not limited to the context of a food or beverage, nutraceutical, pharmaceutical, personal care product, or cosmetic composition. Accordingly, this invention can comprise one or more food, beverage, nutraceutical, pharmaceutical, cosmetic, personal care and various other end-use products or compositions comprising liposome compositions of the sort described herein, such end-use products or compositions as would be known to those skilled in the art made aware of this invention.

### BRIEF DESCRIPTION OF THE DRAWINGS

FIG. 1. (Prior Art). General schematic structure of liposomes. (A) Small or large unilamellar liposomes with a single bilayer structure forming the spherical particle (B) Multiple Vesicular Liposomes comprised of a single large liposomes containing a variety of single (or multiple) liposomes and Multilamellar (onion-type) liposomes composed of concentric layers of membranes.

FIG. 2. Schematic illustration of a principle of formation of double layered liposomes by addition of oppositely charged polymers to stable liposomal preparations that were formed by sonication, microfluidization or high-pressure homogenization.

FIG. 3. Schematic illustration of possible mechanisms involved in formation of double layered liposomes. (A) reversal of surface charge as a function of polymer concentration. (B) Windows of stability for double-layered liposomes as a function of polymer concentration

FIG. 4. Digital images portraying the stability of liposomes in the presence of chitosan. Left: single liposomes. To right, increasing concentrations of chitosan added to liposomal preparations.

FIGS. 5A-C. Micrograph images showing the stability of liposomes in the presence of chitosan. (A) Low concentrations of chitosan leading to large aggregates and phase separation. (B) medium concentrations of chitosan resulting in stable preparations (C) large concentrations of chitosan inducing flocculation.

FIGS. 6A and B. Influence of addition of chitosan (0-0.5 w/v %) to liposomes (0.5 w/v % lecithin, 100 nm) on (A) ζ-potential and (B) mean particle diameter of liposomes.

FIG. 7. Change in ζ-potential of liposomes (100 nm) with concentrations ranging from 0.1 to 1.0 w/v % upon addition of chitosan (0-0.2 w/v %). Insert: Calculated saturation concentration of chitosan from change of liposomal charge.

FIG. 8. Micrographic images (A-D; 100× magnification) and digital images (E) of liposomes (0.5 w/v %, 100 nm mean initial particle size) upon addition of (A) 0% (B) 0.02%, (C) 0.07% and (D) 0.2 wt % chitosan.

FIG. 9. Zeta potential of Liposomes stabilized by chitosan, fish gelatin, b-lactoglobulin and without a coating after manufacturing and after storage for 7 days

FIG. 10. Micrographic images of formation of stable double layered liposomes with chitosan, b-lactoglobulin and fish gelatin. Region (S) denotes the stable regions, region (B) and (D) denote regions of instability where flocculation and aggregation occurs. Region (S) is the structures that are manufactured as part of this technology application.

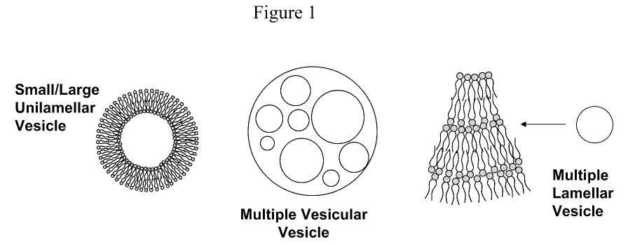

### DETAILED DESCRIPTION OF CERTAIN EMBODIMENTS

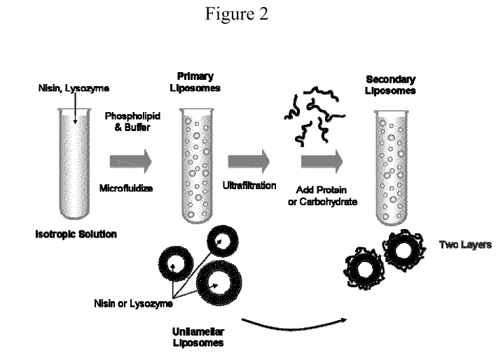

The present invention, as illustrated by several representative embodiments, can be used to overcome various liposome-associated problems. As demonstrated below, this invention provides a multilayered liposome composition and related methods of use by electrostatically depositing one or more oppositely charged polymers on the liposome surface. Such an approach can be employed with any synthetic or naturally-derived bipolymers (e.g., proteins, carbohydrates, etc.) as long as the bipolymer is oppositely charged with respect to the liposome under environmental conditions (e.g., pH, temperature, ionic strength) affecting liposome formation or bipolymer interaction therewith. Depending on the property of the primary lipsome, the deposited secondary bipolymer layer can be cationic or anionic. For example, if the surface charge of the liposome is positive due to the composition of associated phospholipids (e.g., high concentrations of phosphatidylethanolamine), a negatively charged polymer could be added. Alternatively, if the liposome surface charge is negative (as for the majority of liposomal systems), a positively charged compound could be added. FIG. 2 schematically shows one implementation of this invention; a two-step process in which the liposomal preparation is first formed using a conventional method known in the art such as homogenization by high pressure, microfluidization or sonication (or isolation, redispersion then homogenization); and the layer component is then formed by carefully controlled addition of the polymer to the dispersion.

The addition step requires careful selection of appropriate polymers and mixing conditions (e.g., pH, ionic strength, temperature, composition). The molecular weight, charge and environmental conditions can be designed such that polymers adsorb and fully cover the interface and do not destabilize the interfacial layer of the liposome or promote liposome aggregation. In addition, the size and concentration of the liposome can influence the range of conditions in which stable liposomes can be obtained.

Liposome materials and/or lipidic compounds of such liposomes can be isolated or derived from a number of sources known to those skilled in the art, including, but not limited to any land or marine-based animal or plant. For example, such materials and/or compounds are available from various fruits, plant and seed oils (e.g., olive, peanut, palm, soy, etc.), eggs, animal fat, krill, plankton and algae.

Regardless of source or origin, liposomes may be prepared from a wide variety of lipidic compounds, as would be known to those skilled in the art, including but not limited to alkylamines, gangliosides, cardiolipin, and phospholipids. Some of the more widely used lipids include the phospholipids wherein the phosphatidyl portion contains ester groups, for example C₁₄ to C₂₀, saturated or unsaturated, fatty acids. Specifically, liposomes may be prepared from any one or combination of dicetylphosphate, distearoylphosphatidic acid, dipalmitoylphosphatidylcholine, dimyristoylphosphatidylinositol, mono and dialkyl esters of phosphatdylserine, and dioleoylphosphatidylglycerol. In addition, lysophosphatides, wherein the fatty acid ester of C₁ or C₂ has been removed, can be mixed with the above-noted lipids to form liposomes. Other synthetic phosphatidyl compounds include those wherein a sulfonium, phosphonium, or quaternary ammonium polar head moiety has been modified by the addition of hydrocarbon groups, particularly alkyl groups. A range of polar and ionic lipid and/or lipidic compounds, including those representative compounds listed in Table 1, is available from a number of commercial sources, including but not limited to Avanti Polar Lipids, Inc.

TABLE 1
1,2-Distearoyl-sn-Glycero-3-Phosphocholine (DSPC);
1,2-Dioleoyl-sn-Glycero-3-Phosphocholine (DOPC);
1,2-Dimyristoyl-sn-Glycero-3-Phosphoethanolamine (DMPE);
1,2-Dipalmitoyl-sn-Glycero-3-Phosphoethanolamine (DPPE);
1,2-Dioleoyl-sn-Glycero-3-Phosphoethanolamine (DOPE);
1,2-Dimyristoyl-sn-Glycero-3-Phosphate (Monosodium Salt (DMPA);
1,2-Dipalmitoyl-sn-Glycero-3-Phosphate (Monosodium Salt) (DPPA);
1,2-Dioleoyl-sn-Glycero-3-Phosphate (Monosodium Salt) (DOPA);
1,2-Dimyristoyl-sn-Glycero-3-[Phospho-rac-(1-glycerol)] (Sodium Salt)
(DMPG);
1,2-Dipalmitoyl-sn-Glycero-3-[Phospho-rac-(1-glycerol)] (Sodium Salt)
(DPPG);
1,2-Dioleoyl-sn-Glycero-3-[Phospho-rac-(1-glycerol)] (Sodium Salt)
(DOPG);
1,2-Dimyristol-sn-Glycero-3-[Phospho-L-Serine] (Sodium Salt) (DMPS;
1,2-Dipalmitoyl-sn-Glycero-3-[Phospho-L-Serine] (Sodium Salt) (DPPS;)
1,2-Dioleoyl-sn-Glycero-3-[Phospho-L-Serine] (Sodium Salt) (DOPS);
1,2-Dioleoyl-sn-Glycero-3-Phosphoethanolamine-N-(glutaryl) (Sodium
Salt);
1,1′,2,2′-Tetramyristoyl Cardiolipin (Ammonium Salt);
1,2-Dipalmitoyl-sn-Glycero-3-Phosphoethanolamine-N-[Methoxy
(Polyethylene glycol)-2000] (Ammonium Salt);
1,2-Dipalmitoyl-sn-Glycero-3-Phosphoethanolamine-N-[Methoxy
(Polyethylene glycol)-5000] (Ammonium Salt); and
1,2-Dioleoyl-3-Trimethylammonium-Propane (Chloride Salt) (DOTAP)

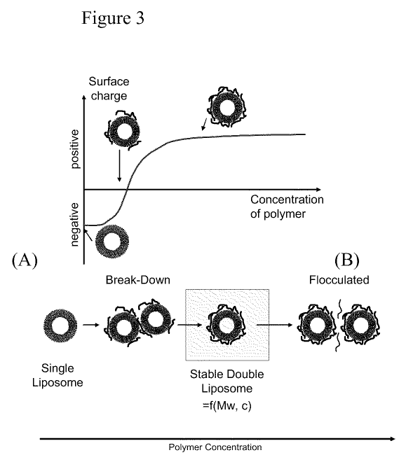

Without limitation to theory or mode of operation, FIG. 3 illustrates a mechanism which may be involved in formation of the double layered liposomes. As additional polymers are added to the liposomal preparations, the surface charge will begin to reverse. Eventually as enough polymer is added, the surface of liposomes will be fully covered and the charge will depend on that of the oppositely added polymer. Correspondingly, stable double layered liposomes will be obtained if enough polymer is added to fully cover the liposomal surfaces. If less polymer is added, destabilization may occur leading to formation of large aggregates or to complete phase separation. Again this process will depend on the molecular properties of the polymer to be added, the properties of the liposome and the environmental conditions.

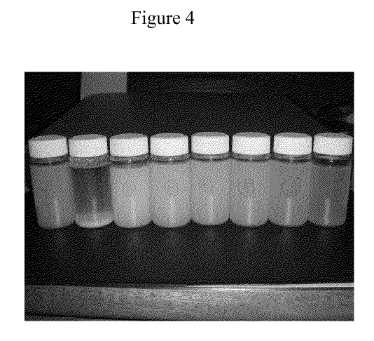

In FIG. 4, digital images of anionic liposomes containing increasing concentrations of cationic chitosan are shown. On the left, a nearly clear solution of liposomes with sizes of 100 nm is shown. Upon addition of small concentrations of chitosan, the system completely destabilizes to form large aggregates that eventually phase separate (sediment). In the presence of more chitosan, solutions become slightly more turbid than original suspension due to adsorption of an additional layer of polymers but do not phase separate. Size measurements of liposomes have shown that their diameter is approx. 200 nm. When much more chitosan is added, flocculation occurs. Table 2 shows an overview over the stable region.

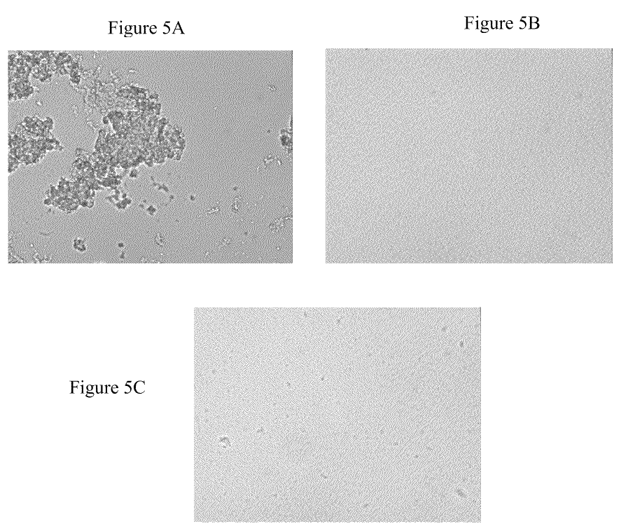

Microscopy confirms the absence of aggregates when appropriate amounts of chitosan are added confirming that stabilized liposomes are prepared (FIG. 5B). Phase separated liposomes with not enough chitosan can be seen in FIG. 5A and liposomes with too much chitosan that are flocculated are shown in FIG. 5C.

TABLE 2
Stability of liposomes in the presence of chitosan.

| | | Liposomes 1% w/v | | Chitosan 0.1% w/v | |
|---|---|---|---|---|---|
| | Sam- | Amount | Final concen- | Amount | Final concen- |
| Comments | ples | (ml) | tration % | (ml) | tration % |
| Brigding | 4 | 14 | 0.7 | 6 | 0.03 |
| flocculation | 5 | 12 | 0.6 | 8 | 0.04 |
| | 6 | 10 | 0.5 | 10 | 0.05 |
| stable | 7 | 8 | 0.4 | 12 | 0.06 |
| | 8 | 6 | 0.3 | 14 | 0.07 |
| Depletion | 9 | 4 | 0.2 | 16 | 0.08 |
| flocculation | 10 | 2 | 0.1 | 18 | 0.09 |

(A) Low concentrations of chitosan leading to large aggregates and phase separation. (B) medium concentrations of chitosan resulting in stable preparations (C) large concentrations of chitosan inducing flocculation

More particularly, for purposes of further characterization, liposome suspensions were produced by homogenizing 1% soy lecithin in acetate buffer (0.1 M, pH 3). Cationic chitosan (Mw˜200 kDa) solutions were mixed with anionic liposome suspensions (d≈100 and 200 nm) and the effect of phospholipid concentration, chitosan concentration and liposome size on the properties of the particles formed was determined. The particle size and charge (ζ-potential) were measured using dynamic light scattering and particle electrophoresis.

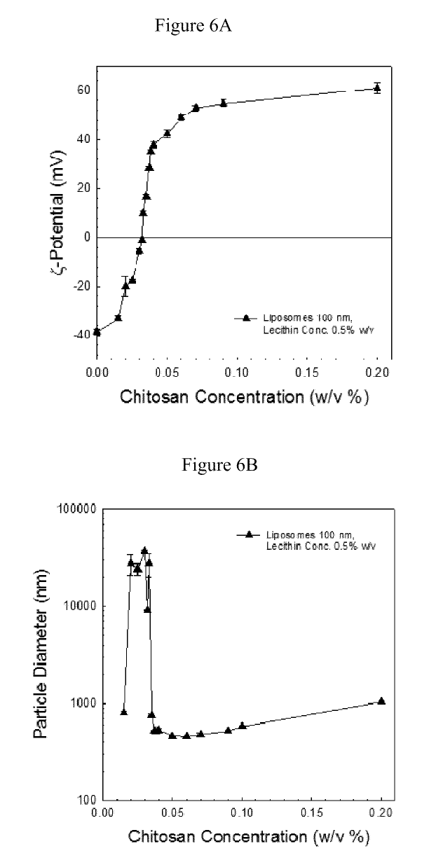

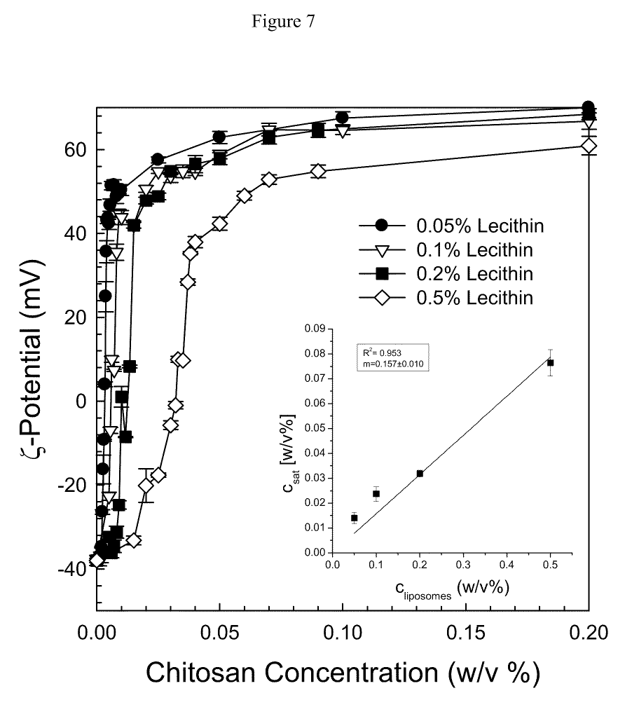

The interaction between chitosan and liposomes (d≈98 nm) was monitored by measuring the electrical charge and mean diameter of the particles in mixed liposome suspensions at pH 3 (FIGS. 6 and 7). ζ-potential measurements indicated that the uncoated liposomes were highly anionic (−38 Mv), whereas the chitosan molecules were highly cationic (+85 Mv). The surface charge of the particles in liposome suspensions (0.5% lecithin) increased from −38 Mv to +60 Mv with addition of chitosan (0-0.2%) (FIG. 6A). The net charge on the particles was zero after addition of approximately 0.032 w/v % chitosan indicating that charge neutralization occurred at this liposome-to-chitosan composition. The observed changes in surface charge suggest that chitosan molecules adsorbed to the surfaces of liposomes until the liposomal membrane was fully covered with chitosan molecules thereby preventing further adsorption.

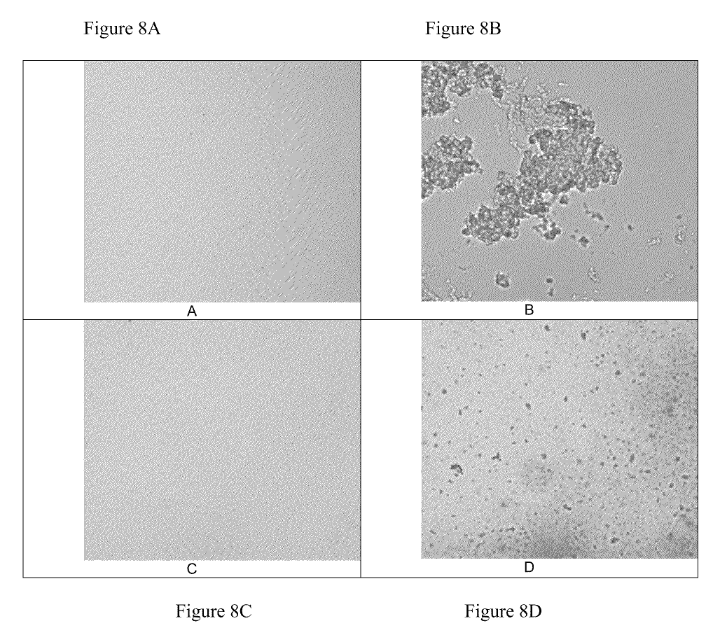

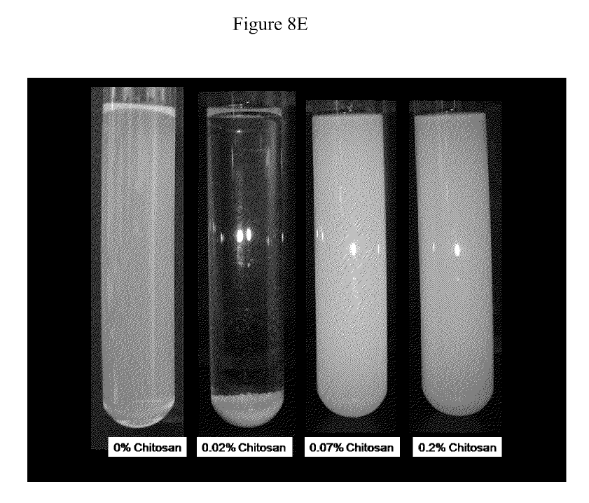

The mean diameter of the particles in the suspensions was highly dependent on the concentration of chitosan added to the system (FIG. 6B). The particle diameter increased from around 98 nm in the absence of chitosan to well above 10,000 nm in the presence of low amounts of added chitosan (0.02 to 0.04 w/v %), indicating the formation of large aggregated structures (FIG. 6B). These aggregates eventually phase separated and formed a precipitate at the bottom of the test tube (FIG. 8). The range of chitosan concentrations where large aggregates was formed corresponded to the surface charge of the particles changing from approximately −20 Mv to +20 Mv, which suggested that this type of aggregation was caused by charge neutralization or possibly bridging (FIG. 6A). At chitosan concentrations above 0.04 wt %, the particle diameter decreased to below about 300 nm to reach a minimum value at a chitosan concentration of 0.07 w/v %. Further addition of chitosan led to steady increases in particle diameter, e.g., the particle diameter was approximately 900 nm after addition of 0.2 w/v %.

The impact of liposome concentration and diameter on the formation and stability of coated liposomes was determined. The ζ-potential and mean particle diameter of liposomal dispersions containing from 0.05 to 0.5 w/v % lecithin upon addition of 0 to 0.2 w/v % chitosan was measured (FIG. 2). The charge of liposomes increased from −40 Mv to above +60 Mv with addition of chitosan regardless of the initial lecithin concentration (FIG. 7). However more chitosan was required to cause charge reversal with increasing concentrations of lecithin. In addition, the increase in the surface charge occurred over a wider range of chitosan concentrations. For example, at a lecithin concentration of 0.05 w/v %, the charge increased from −20 Mv to +20 Mv when the chitosan concentration was increased from 0.007 to 0.012 w/v % while at a lecithin concentration of 0.5% the chitosan concentration had to be increased from 0.02 to 0.04 w/v % to cause a similar change in ζ-potential.

FIG. 8 shows the optical microscopy (A-D) and digital (E) images of uncoated (0 w/v % chitosan) and coated liposomes (0.07 w/v % chitosan) after storage for 45 days at a liposome concentration of 0.5 w/v %. While uncoated liposomes were stable after manufacturing, they eventually broke down to form large needle shaped precipitates that began to sediment to the bottom of the test tube. Conversely, chitosan-coated liposomes remained stable after storage for 45 days: no phase separate was observed and the particle size distribution did not change appreciably, e.g., the mean diameter changed from 250 to 260 nm from day 0 to day 45. Thus, chitosan-coated liposomes were significantly more stable to long-term storage than uncoated liposomes. In general, liposomes have a tendency to merge to form larger liposomes over time that may eventually lead to phase separation. The molecular geometry of polar lipids in a bilayer often means that the most thermodynamically stable configuration is a flat monolayer with zero curvature. Without limitation to any one theory or mode of operation, the merging of vesicles is due to the flexibility and natural undulations of the bilayer and the absence of sufficient electrostatic repulsive forces that could prevent the liposomes from coming into close contact. Coating of liposomes with bipolymers, in accordance with this invention, thus greatly improves ability to stabilize liposomes. This mechanism will also improve stability under a variety of temperatures and in a variety of pH environments as well as in the presence of salts.

Other embodiments can be illustrated by electrostatic deposition of a variety of oppositely charged bipolymers on primary liposomes. Such evaluation included compound concentrations required to produce stable dispersions of coated food grade liposomes by study of vesicle diameter and ζ-potential. To obtain primary liposomes (˜100 nm), a lecithin solution was passed trough a microfludizer three times at 7000 psi (Model 11OL, Microfludizer, Microfludics, Newton Mass.). These solutions were stored at 4° C. before using, but no longer than 24 h. Various liposome preparation techniques are well-known to those skilled in the art, such methods together with a range of lipidic compounds useful therewith as described in U.S. Pat. No. 5,139,803, the entirety of which is incorporated herein by reference.

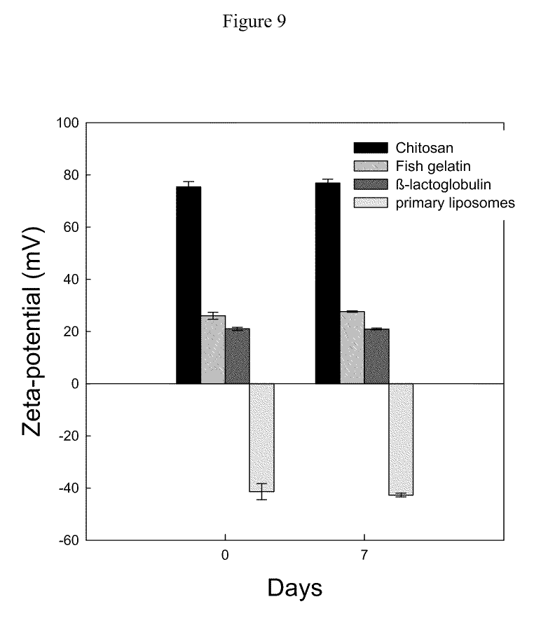

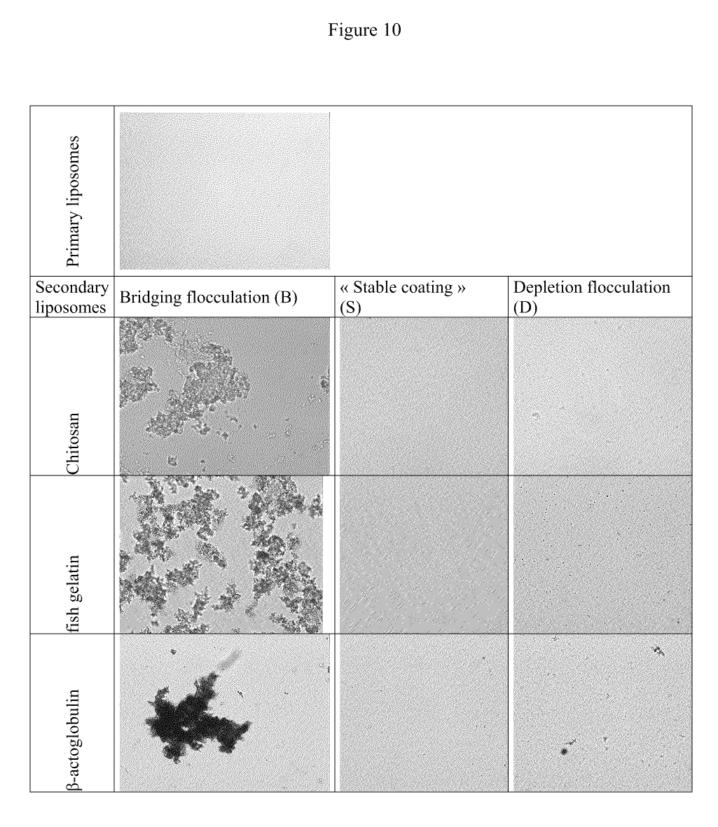

An amount of primary liposomes was added to solutions containing a variety of polymer concentration (0.025-5% v/w) at room temperature (20° C.), under continuous high stirring for 1 min. Vesicle size were measured by Dynamic Light Scattering (DLS), with a Zetasizer (nanoseries nanoZS, model ZEN3600, Malvern Instrument Limited, Worcestershire, UK). Surface charge of primary and secondary liposomes were measured with a Zetasizer (nanoseries nanoZS, model ZEN3600, Malvern Instrument Limited, Worcestershire, UK). FIG. 9 shows results of a zeta potential measurement of liposomes stabilized by a variety of oppositely charged bipolymers, including fish gelatin, b-lactoglobulin and chitosan. The zeta potential measurements show that liposomal charge was successfully reversed indicating full coverage of liposomal surfaces by the different polymers, i.e., the surface charge reverted from −38 Mv to +70 Mv for chitosan, to +25 Mv for fish gelatin and to +20 Mv for b-lactoglobulin. Similarly to previously described stability measurements, liposomes covered with the different bipolymers were stable within certain bipolymer concentrations. (See FIG. 10.)

As demonstrated above, this invention can be used to stabilize liposomes with any bipolymer oppositely charged with respect to the liposome, making it possible to produce temperature, salt and pH stable liposomes that do not leak, resist mechanical stresses and abuse conditions better than current liposomal preparations. In addition, such interactions can be addressed by using more economical polymers instead of expensive, purified phospholipids components. With increased structural robustness, liposomal use can be extended. In addition, with the present invention, liposome systems could be made entirely from generally recognized as safe (GRAS) food grade ingredients, such as proteins, polysaccharides and phospholipids, and could therefore be used in edible products without the need for costly and time-consuming FDA GRAS regulatory approval.

Illustrating use of the present invention to address the various such related concerns, consider liposomes as a delivery system for bioactive lipids such as conjugated linoleic acid and omega-3 and omega-6 fatty acids. It has been envisioned, for example, liposomes from marine phospholipids could be used to deliver omega-3 fatty acids to foods without altering the clarity or color of the food product. However, liposomes currently used are negatively charged and attract positive iron ions, leading to accelerated lipid oxidation, discoloration and turbidity. The present invention can be used to alter liposome surface charge. For instance, at pH 3, liposomes can be coated with a protein such as whey protein isolate (WPI). Electrostatic deposition of such a bipolymer component provides a positive surface charge to the liposome composition, repels iron ions, significantly increases oxidative stability of the resulting composition and enhances its utility as a source of bioactive agent.

While the preceding illustrates a liposome composition including an omega-3 fatty acid or associated fish oil, the liposome compositions of this invention can incorporate any substance soluble or miscible in either a liposome core or an associated lipid bilayer. Without limitation, oxidizable lipids, common in the food industry can, by way of example be incorporated into a lipid bilayer. Such oxidizable lipids include, but are not limited to eicosatetraenoic acid, eicosapentaenoic acid, docosahexaenoic acid, oleic acid, linoleic acid, linolenic acid and various arachidonic acids. Without limitation, such a lipid component can be present in a lipidic bilayer in the range from about 10 to about 30% by weight of the total lipid content in the liposome composition. Other active agents which can be associated with such a liposome or bilayer thereof include various nutrients, vitamins, carotenoids, glycerol, acidulants, preservatives, flavorants, colorants, flavor oils, food oils antioxidants, antimicrobials, ultra-violet light absorbers and tropicalizing agents for confectionary coatings and compounds.

Accordingly, this invention is applicable in products that currently use liposomes to deliver functional ingredients or active agents, such ingredients/agents, including but not limited to antioxidants, antimicrobials, pharmaceutically active compounds or either need to be microbially stable (foods, nutraceuticals or pharmaceuticals) or are geared towards combating microbial infections (medical, pharmaceuticals and personal care products). For example, to illustrate the latter, deodorants combating microbial outbreaks associated with sweat inducing environments are among such compositions which can incorporate the stabilized liposome compositions of this invention.

The preceding non-limiting examples and data illustrate various aspects and features relating to the compositions and/or methods of the present invention, including the preparation of stabilized liposome compositions, as are available through the methodologies described herein. In comparison with the prior art, the present compositions and methods provide results and data which are surprising, unexpected and contrary thereto. While the utility of this invention is illustrated through the use of various lipidic compounds, liposomes, active agents and biopolymer components, it will be understood by those skilled in the art that comparable results are obtainable with various other lipidic compounds, liposomes, active agents and biopolymer components, as are commensurate with the scope of this invention.

We claim:

1. A stabilized liposome composition comprising a liposome comprising a lipidic compound, said liposome comprising a hydrophilic peripheral surface component, at least a portion of said peripheral surface component comprising a net charge; and a first biopolymer component about said liposome, at least a portion of said biopolymer component comprising a net charge opposite the net charge of said liposome peripheral surface component.

2. The composition of claim 1 wherein a said lipidic compound is selected from phospholipid compounds, phosphatide compounds, and combinations of said compound.

3. The composition of claim 1 wherein a said first biopolymer component is selected from proteins, polysaccharides and combinations thereof.

4. The composition of claim 1 wherein said liposome peripheral surface component comprises a net negative charge and a said first biopolymer component comprises a net positive charge.

5. The composition of claim 4 comprising a liposome comprising a soy lecithin and one of a chitosan and a whey protein isolate component thereabout.

6. The composition of claim 1 wherein said lipipidic and first biopolymer components are food grade.

7. The composition of claim 1 comprising a second biopolymer component about said first biopolymer component, at least a portion of said second biopolymer component comprising a net charge opposite the net charge of said first biopolymer net charge.

8. The composition of claim 7 wherein said first biopolymer component has a net positive charge, and said second biopolymer component has a net negative charge.

9. The composition of claim 8 comprising one of a chitosan and a whey protein isolate component, and a pectin component thereabout.

10. The composition of claim 7 wherein said lipidic compound and first and second biopolymer components are food grade.

11. The composition of claim 1 wherein said liposome comprises a configuration selected from unilamellar and multi-lamellar bilayer configurations.

12. The composition of claim 11 wherein a said bilayer comprises an active agent at least partially miscible therein.

13. The composition of claim 12 wherein said active agent is selected from an antioxidant, an antimicrobial, a pharmaceutical and combinations thereof.

14. The composition of claim 1 wherein said liposome core comprises an aqueous medium comprising an active agent at least partially miscible therein.

15. The composition of claim 14 wherein said active agent is selected from an antioxidant, an antimicrobial, a pharmaceutical and combinations thereof.

16. The composition of claim 1 incorporated into an end-use product.

17. A composition comprising a liposome comprising a lipidic compound, said liposome comprising a hydrophilic peripheral surface component, at least a portion of said surface component comprising a net charge; a first biopolymer component about said liposome, at least a portion of said first biopolymer component comprising a net charge opposite said liposome peripheral surface component net charge; and a second biopolymer component about said first biopolymer component, at least a portion of said second biopolymer component comprising a net charge opposite said first biopolymer component net charge.

18. The composition of claim 17 wherein a said lipidic compound is selected from phospholipid compounds, phosphatide compounds, and combinations thereof.

19. The composition of claim 17 wherein first and second biopolymer components are independently selected from proteins, polysaccharides and combinations thereof.

20. The composition of claim 19 wherein each of said lipid compound and said first and second biopolymer components are food grade.

21. The composition of claim 20 wherein said first biopolymer component is selected from a chitosan and a whey protein isolate, and said second biopolymer component is a pectin.

22. The composition of claim 17 comprising at least one of a liposome bilayer configuration and an aqueous core component.

23. The composition of claim 22 wherein an active component is at least partially miscible in at least one of said bilayer configuration and said core component.

24. The composition of claim 23 wherein said active agent is selected from an antioxidant, an antimicrobial, a pharmaceutical and combinations thereof.

25. A method of preparing a liposome composition, said method comprising;
providing an aqueous medium comprising a liposome comprising a lipidic compound, said liposome comprising a hydrophilic peripheral surface component, at least a portion of said peripheral surface component comprising a net charge;
contacting said liposome with a first biopolymer component, at least a portion of said first biopolymer component comprising a net charge opposite said peripheral surface component net charge; and
contacting said first biopolymer component with a second biopolymer component, at least a portion of said second biopolymer component comprising a net charge opposite said first biopolymer component net charge.

26. The method of claim 25 wherein said liposome has a ζ-potential of said net charge, said first biopolymer component is in an amount at least partially sufficient to provide said composition a ζ-potential of said opposite charge, and said second biopolymer component is in an amount at least partially sufficient to provide said composition a ζ-potential of said opposite charge.

27. The method of claim 25 wherein at least one biopolymer component net charge is provided by adjusting medium pH.

28. The method of claim 27 wherein said biopolymer components are independently selected from proteins, polysaccharides and combinations thereof.

29. The method of claim 28 wherein a said biopolymer component comprises a protein, and said medium pH is lowered below the isolelectric point of said protein.

30. The method of claim 25 comprising iterative biopolymer contacts, each said biopolymer component independently selected from proteins, polysaccharides and combinations thereof.

31. A method of using a biopolymer component to oxidatively stabilize a lipsome composition, said method comprising;
providing a liposome composition comprising a lipidic compound, said liposome comprising a hydrophilic peripheral surface component, at least a portion of said peripheral surface component comprising a net negative charge, said liposome comprising an oxidazible active agent therein; and
coupling said liposome composition with a biopolymer component, at least a portion of said biopolymer component comprising a net positive charge,
said liposome composition comprising a net positive charge at least partially sufficient to at least partially repel a positively charged oxidizing agent.

32. The method of claim 31 wherein said composition is in a medium comprising an oxidizing agent is selected from metal cations.
